(19) United States
(12) Patent Application Publication
(10) Pub. No.: US 2006/0047219 A1
(43) Pub. Date: Mar. 2, 2006

(54) Cervical biopsy system

(76) Inventors: Dingane Baruti, Columbus, GA (US); Mary A. Dean, Bakersfield, CA (US)

Correspondence Address: Michael S. Neustel, Suite No.4, 2534 South University Drive, Fargo, ND, 58103, US

(21) Appl. No.: 10/933,768

(22) Filed: Sep. 2, 2004

(51) Int. Cl. A61B 10/00; A61B 17/32

(52) U.S. Cl. 600/564; 606/167

(57) ABSTRACT

A cervical biopsy system for improving the quality and efficiency of cervical biopsies. The cervical biopsy system includes an elongated housing with a handle attached to one end, a punch attached to an opposite end of the housing, a retrieval blade movably positioned within the punch for severing a biopsy tissue, and a trigger member mechanically connected to the retrieval blade for manipulating the retrieval blade within the punch. The punch is inserted into the inner wall of the uterine cervix for cutting around a uniform piece of biopsy tissue. The retrieval blade is manipulated to sever the biopsy tissue from the uterine cervix and the user is able to withdraw the biopsy tissue for medical testing.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to cervical biopsy devices and more specifically it relates to a cervical biopsy system for improving the quality and efficiency of cervical biopsies.

2. Description of the Related Art

Cervical biopsy devices have been in use for years for retrieving uterine cervix tissue for medical testing (e.g. cancer testing). It is important during a cervical biopsy to recover all layers of cervical epithelium within the biopsy tissue which are required for an accurate medical analysis.

Typically, during a colposcopy examination the cervix is drenched with acetic acid which turns certain areas a white color representing dysplasia which need to be biopsied. Long neck biopsy forceps are typically used to collect a portion of the abnormal cervix tissue for pathologic examination. U.S. Pat. No. 6,309,404 teaches a modern version of the biopsy forceps which has a pair of cutting jaws (upper jaw and lower jaw) for grasping and severing the biopsy tissue from the uterine cervix. The jaws of the biopsy forces are utilized to cut and tear the biopsy tissue from the inner wall of the uterine cervix. Dermal punches have also been utilized for years for removing tissue samples from the skin of a patient and are basically comprised of a tubular blade structure.

One of the main problems with conventional cervical biopsy devices is that they do not adequately recover all layers of cervical epithelium within the biopsy tissue which are required for an accurate medical analysis. One of the main reasons for inadequate biopsy tissue samples is the poor contact between the biopsy instrument and the cervix. Physicians sometimes must have the patient cough during the biopsy or twisting the forceps to obtain better tissue contact. When the biopsy tissue is inadequate, the patient is usually given the option of a repeat colposcopy in 3-4 months (time required for the cervical inflammation to quell) or to proceed with a minor surgical procedure wherein a larger portion of the cervix is removed. This can result in undesirable and worrisome delays for the patient.

### BRIEF SUMMARY OF THE INVENTION

In view of the foregoing disadvantages inherent in the known types of cervical biopsy devices now present in the prior art, the present invention provides a new cervical biopsy system construction wherein the same can be utilized for improving the quality of cervical biopsies.

The general purpose of the present invention, which will be described subsequently in greater detail, is to provide a new cervical biopsy system that has many of the advantages of the cervical biopsy devices mentioned heretofore and many novel features that result in a new cervical biopsy system which is not anticipated, rendered obvious, suggested, or even implied by any of the prior art cervical biopsy devices, either alone or in any combination thereof.

To attain this, the present invention generally comprises an elongated housing with a handle attached to one end, a punch attached to an opposite end of the housing, a retrieval blade movably positioned within the punch for severing a biopsy tissue, and a trigger member mechanically connected to the retrieval blade for manipulating the retrieval blade within the punch. The punch is inserted into the inner wall of the uterine cervix for cutting around a uniform piece of biopsy tissue. The retrieval blade is manipulated to sever the biopsy tissue from the uterine cervix and the user is able to withdraw the biopsy tissue for medical testing.

There has thus been outlined, rather broadly, the more important features of the invention in order that the detailed description thereof may be better understood, and in order that the present contribution to the art may be better appreciated. There are additional features of the invention that will be described hereinafter and that will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a cervical biopsy system that will overcome the shortcomings of the prior art devices.

A second object is to provide a cervical biopsy system for improving the quality of cervical biopsies.

Another object is to provide a cervical biopsy system that provides a uniform core of cervical tissue.

An additional object is to provide a cervical biopsy system that increases the efficiency and accuracy of cervical dysplasia grading.

A further object is to provide a cervical biopsy system that does not require physicians to remove an excessive amount of cervical tissue to ensure that a full thickness sample is obtained.

Another object is to provide a cervical biopsy system that does not require twisting or other manipulation of the cervical biopsy tool to ensure good tissue interface.

Other objects and advantages of the present invention will become obvious to the reader and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is a side view of the present invention.

FIG. 2 is a side cutaway view of the present invention.

FIG. 3 is a side cutaway view of the present invention with the retrieval blade fully rotated within the punch.

FIG. 4 is a magnified side cutaway view of the punch.

FIG. 5a is a side cutaway view of the punch distally spaced from an inner wall of the uterine cervix.

FIG. 5b is a side cutaway view of the punch extended into the inner wall of the uterine cervix.

FIG. 5c is a side cutaway view of the punch extended into the inner wall of the uterine cervix with the retrieval blade partially rotated.

FIG. 5d is a side cutaway view of the punch extended into the inner wall of the uterine cervix with the retrieval blade fully rotated thereby completely severing the biopsy tissue.

FIG. 5e is a side cutaway view of the punch removed from the inner wall of the uterine cervix with the biopsy tissue contained within the punch.

FIG. 6 is a side view of the retrieval blade.

FIG. 7 is a front view of the retrieval blade.

FIG. 8 is a cross sectional view taken along line 8-8 of FIG. 7.

FIG. 9 is a cross sectional view taken along line 9-9 of FIG. 6.

FIG. 10 is a cross sectional view taken along line 10-10 of FIG. 1.

FIG. 11 is an end view of the punch.

### DETAILED DESCRIPTION OF THE INVENTION

A. Overview

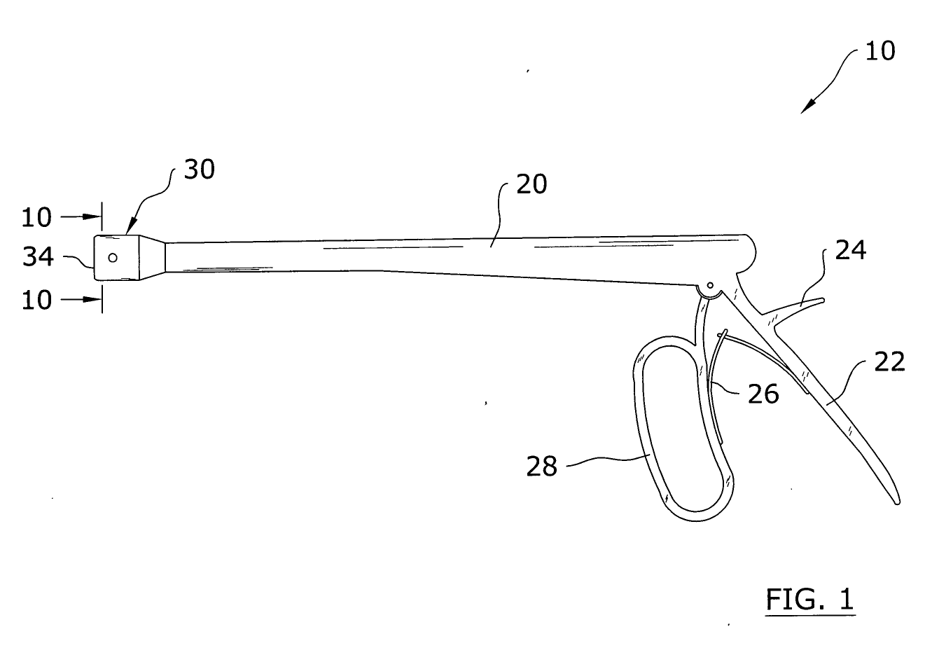

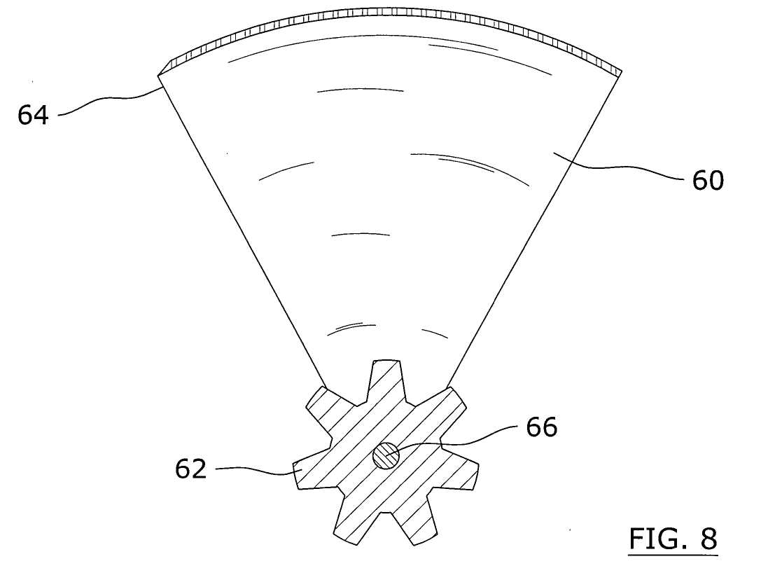

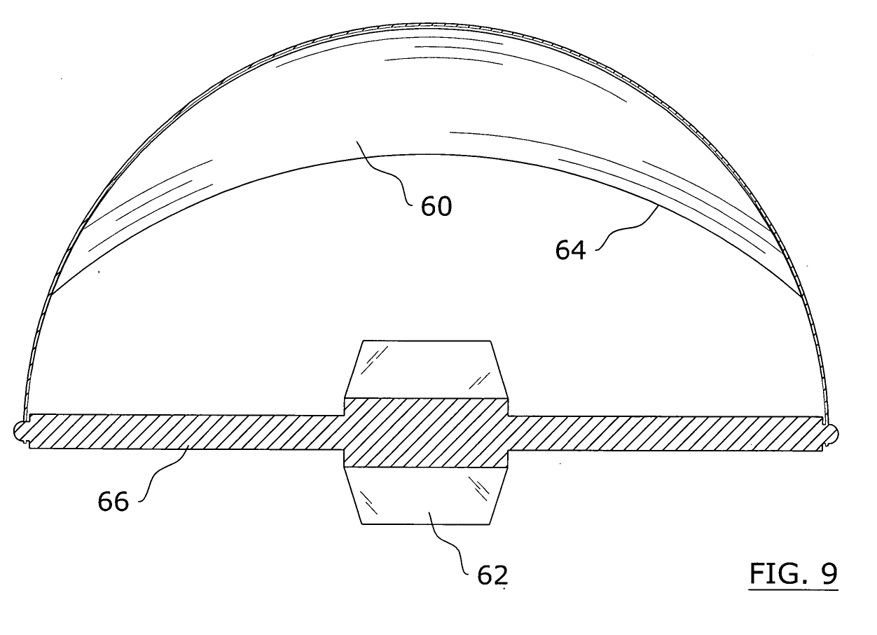

Turning now descriptively to the drawings, in which similar reference characters denote similar elements throughout the several views, FIGS. 1 through 11 illustrate a cervical biopsy system 10, which comprises an elongated housing 20 with a handle 22 attached to one end, a punch 30 attached to an opposite end of the housing 20, a retrieval blade 60 movably positioned within the punch 30 for severing a biopsy tissue 14, and a trigger member 28 mechanically connected to the retrieval blade 60 for manipulating the retrieval blade 60 within the punch 30. The punch 30 is inserted into the inner wall of the uterine cervix 12 for cutting around a uniform piece of biopsy tissue 14. The retrieval blade 60 is manipulated to sever the biopsy tissue 14 from the uterine cervix 12 and the user is able to withdraw the biopsy tissue 14 for medical testing. The present invention may also be utilized for retrieval of biopsy tissue 14 from areas of the human body other than the uterine cervix 12 even though the discussion below is directed towards this area of the human body.

B. Punch

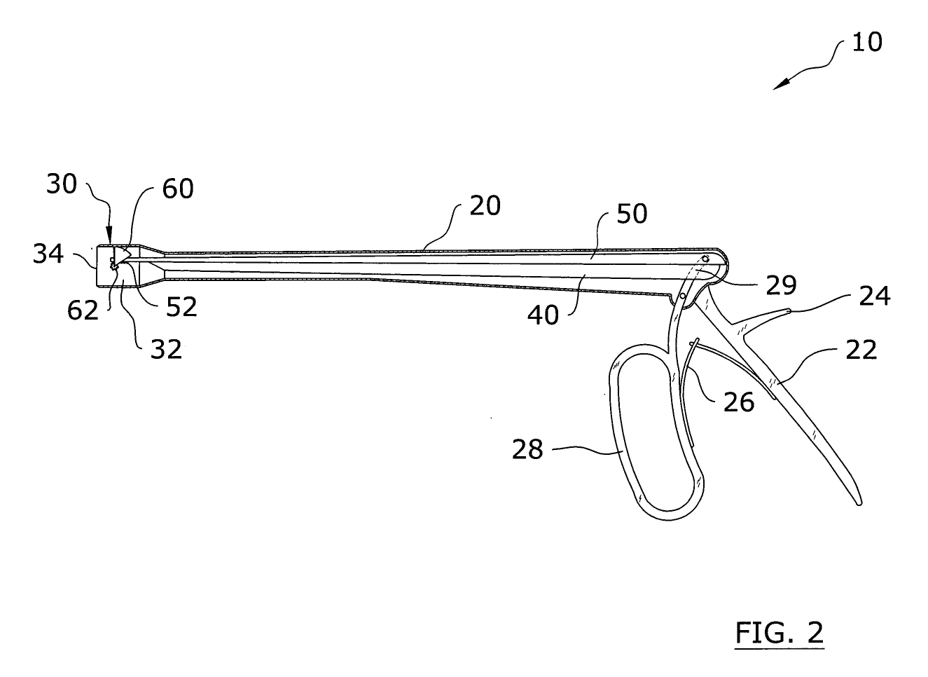

The punch 30 has a tubular structure as illustrated in FIGS. 2 through 5e of the drawings. The punch 30 may be comprised of any dermal punch structure used in the medical industry and other industries as can be appreciated. The punch 30 has a cutting edge 34 and an interior cavity 32 for cutting and receiving the biopsy tissue 14 from the uterine cervix 12. The cutting edge 34 is formed to allow for sufficient cutting and penetration to create a substantially uniform biopsy tissue 14 sample.

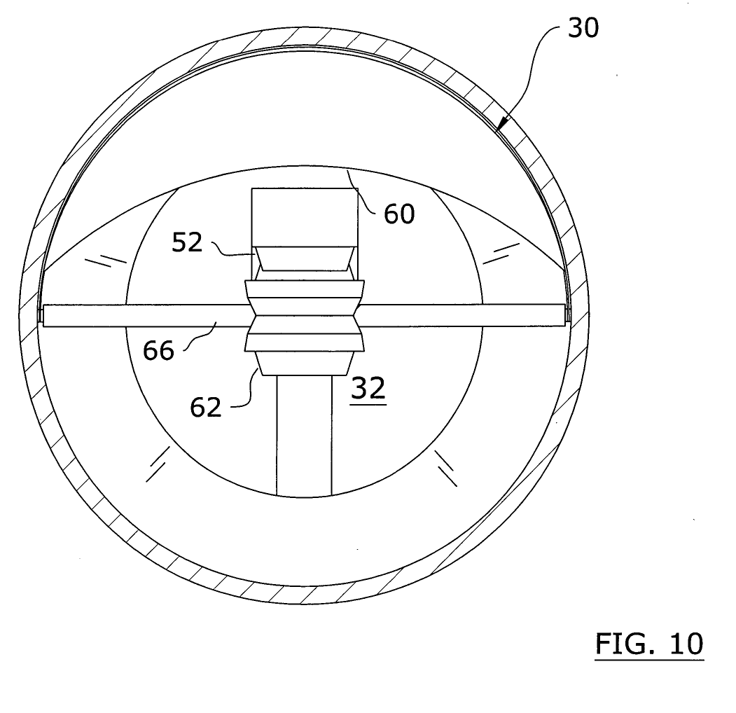

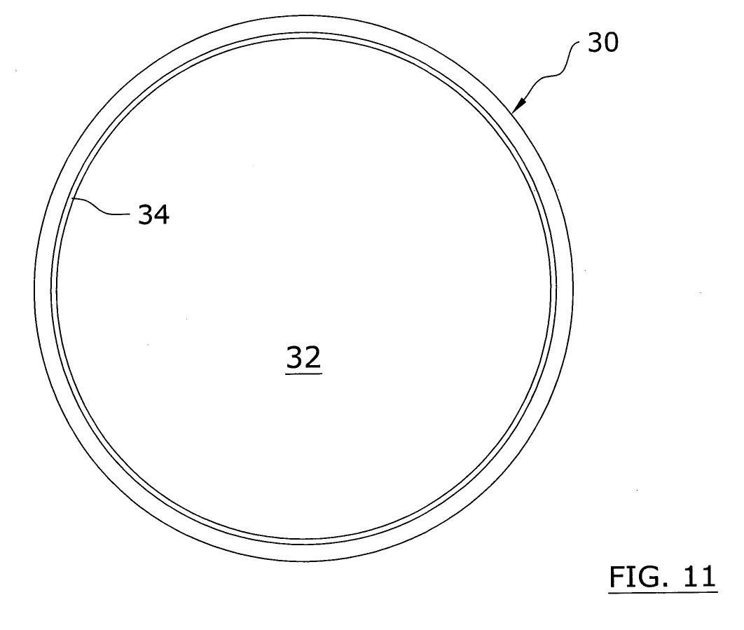

The punch 30 preferably has a circular cross sectional area for cutting a circular area of the uterine cervix 12 as shown in FIGS. 10 and 11 of the drawings. The cutting edge 34 of the punch 30 also preferably has a circular shape as shown in FIG. 11 of the drawings. The diameter of the punch 30 may vary depending upon the size of the sample of biopsy tissue 14 desired. The length of the punch 30 may also vary depending upon the thickness of the sample of biopsy tissue 14 desired. It can be appreciated that the punch 30 may have various other cross sectional shapes and sizes.

C. Retrieval Blade

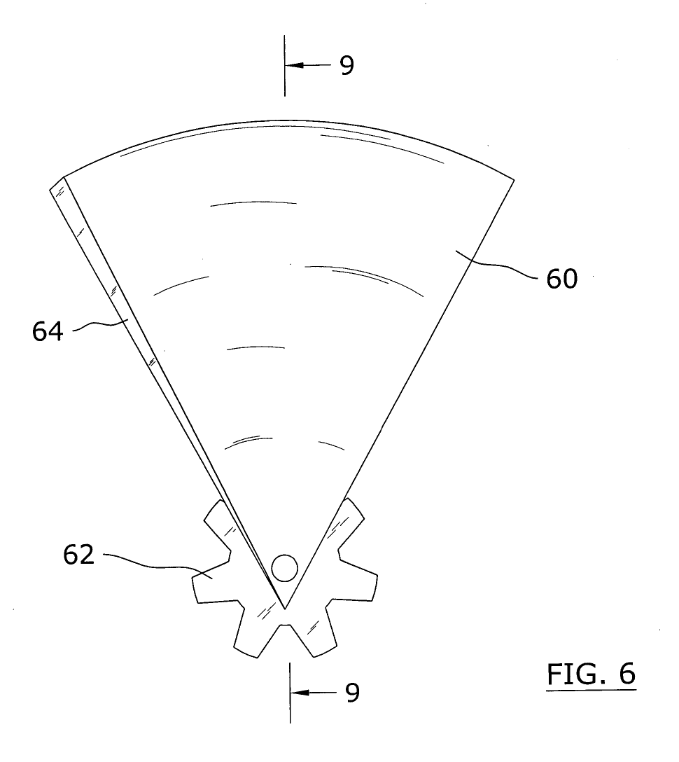

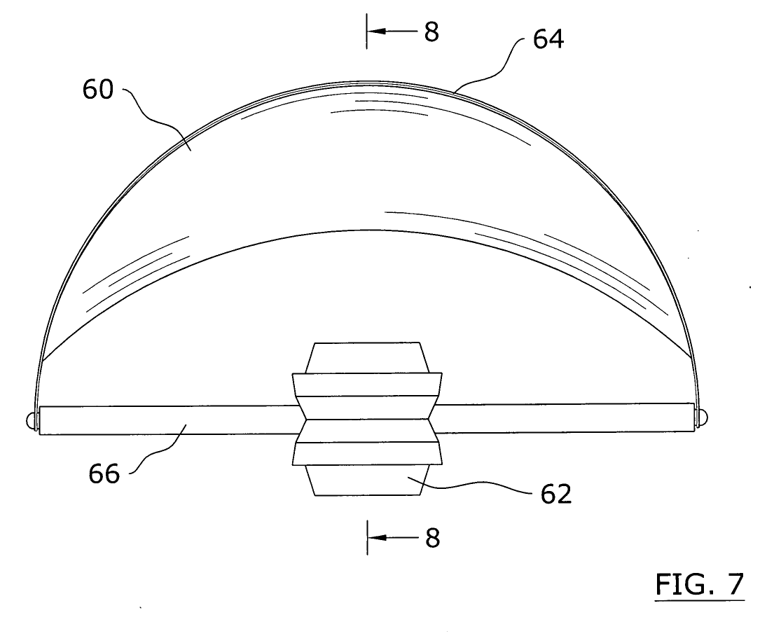

The retrieval blade 60 is movably positioned within the interior cavity 32 of the punch 30 for severing a biopsy tissue 14 penetrated by the punch 30 as shown in FIGS. 2-5e, 10, and 11 of the drawings. The retrieval blade 60 includes a blade edge 64 that is designed for cutting the sample of biopsy tissue 14 as shown in FIGS. 6 and 7 of the drawings.

The retrieval blade 60 preferably has a curved C-shaped structure as best illustrated in FIGS. 7, 9, 10 and 11 of the drawings. It can be appreciated that the retrieval blade 60 may have various other shapes and configurations that are capable of severing the sample of the biopsy tissue 14.

A shaft member 66 preferably extends along a rotation axis of the retrieval blade 60 and is attached to the retrieval blade 60 as illustrated in FIG. 7 of the drawings. The shaft member 66 is rotatably attached to the punch 30 as illustrated in FIGS. 10 and 11 of the drawings. The shaft member 66 is preferably substantially transverse with respect to a longitudinal axis of the punch 30 as best illustrated in FIGS. 10 and 11 of the drawings.

As best shown in FIG. 7 of the drawings, a gear 62 or similar structure is preferably attached to the shaft member 66. The gear 62 mechanically engages the teeth 52 of the arm member 50 for rotating the retrieval blade 60 from an open position to a closed position.

D. Housing

An elongated housing 20 is preferably attached to the punch 30 as shown in FIGS. 1 through 5e of the drawings. The elongated housing 20 is sufficient in length of to reach various locations within the uterine cervix 12. In addition, the width of the elongated housing 20 is preferably narrow enough to allow for sufficient movement within the uterine cervix 12. As shown in FIGS. 2 through 5e of the drawings, the elongated housing 20 is preferably comprised of a tubular structure for movably receiving the arm member 50 within. The housing 20 may be comprised of various other shapes, structures and configurations other than shown in the attached drawings as can be appreciated.

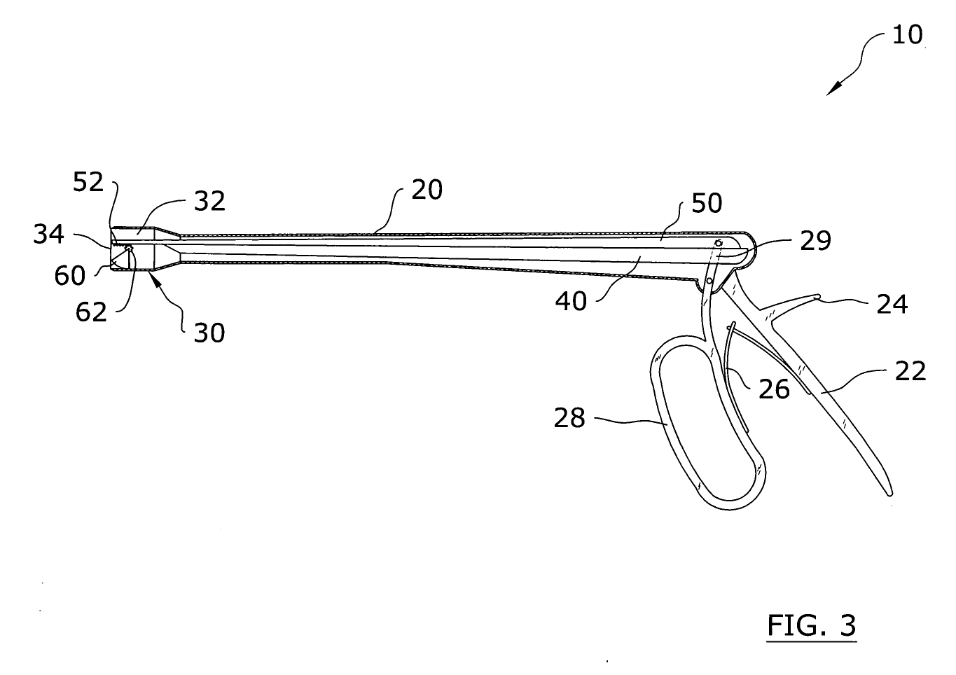

A guide member 40 or similar structure is preferably attached within the housing 20 for guiding the arm member 50 along a longitudinal path within the housing 20. The guide member 40 may extend along a substantial length of the arm member 50 as shown in FIGS. 2 and 3 of the drawings.

As shown in FIGS. 1 through 3 of the drawings, a handle 22 is preferably attached to the elongated housing 20 opposite of the punch 30. The handle 22 is formed for easy grasping by a physician for manipulating the punch 30 within the uterine cervix 12. A thenar brace 24 is preferably attached to the handle 22 as further shown in FIGS. 1 through 3 of the drawings.

A trigger member 28 or similar mechanical structure is mechanically connected to the retrieval blade 60 for manipulating the retrieval blade 60 within the punch 30 as shown in FIGS. 1 through 3 of the drawings. The trigger member 28 may have various configurations and structures other than shown in the attached drawings. The trigger member 28 is pivotally connected to the elongated housing 20 near the handle 22 with a connector member 29 extending past the pivot point as shown in FIGS. 2 and 3 of the drawings. The connector member 29 is mechanically connected to the retrieval blade 60 by the elongated arm member 50 as further shown in FIGS. 2 and 3 of the drawings. It can be appreciated that motorized versions of the present invention may be utilized.

A bias member 26 (e.g. spring) is preferably positioned between the handle 22 and the trigger member 28 for providing a separating force between thereof as shown in FIGS. 1 through 3 of the drawings. The bias member 26 allows the trigger member 28 and the retrieval blade 60 to return to the open position accordingly.

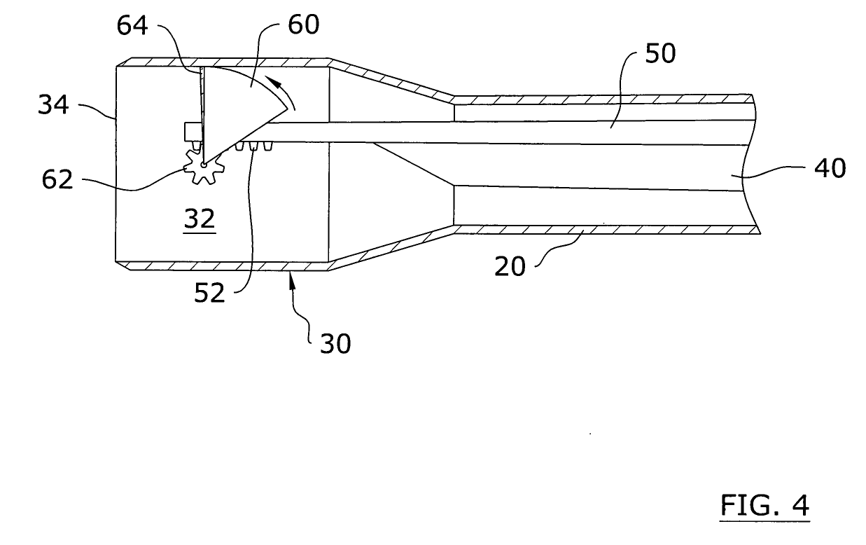

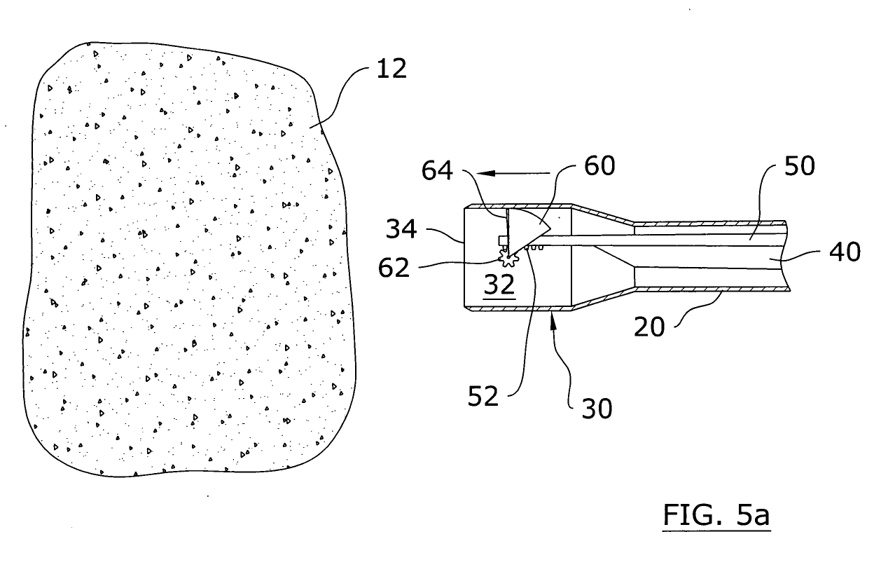

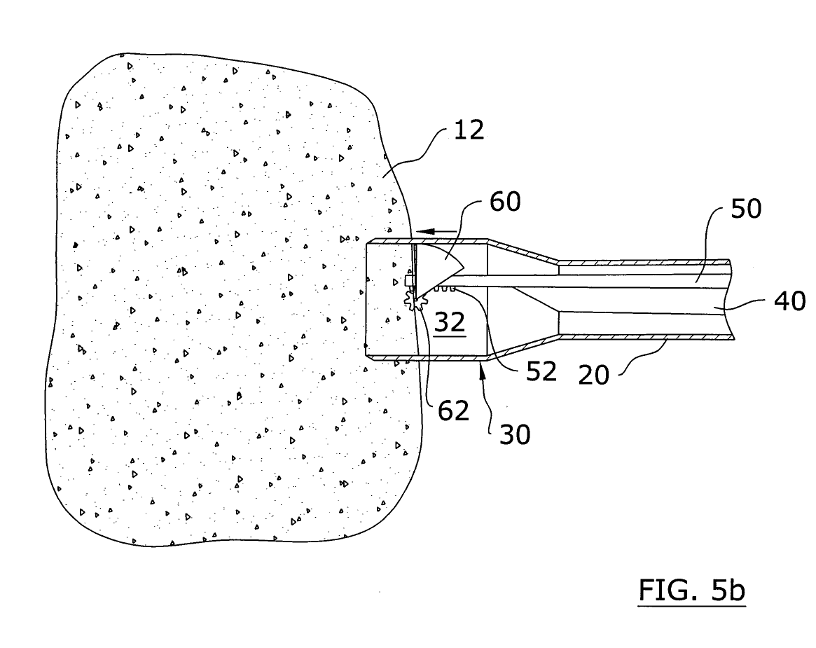

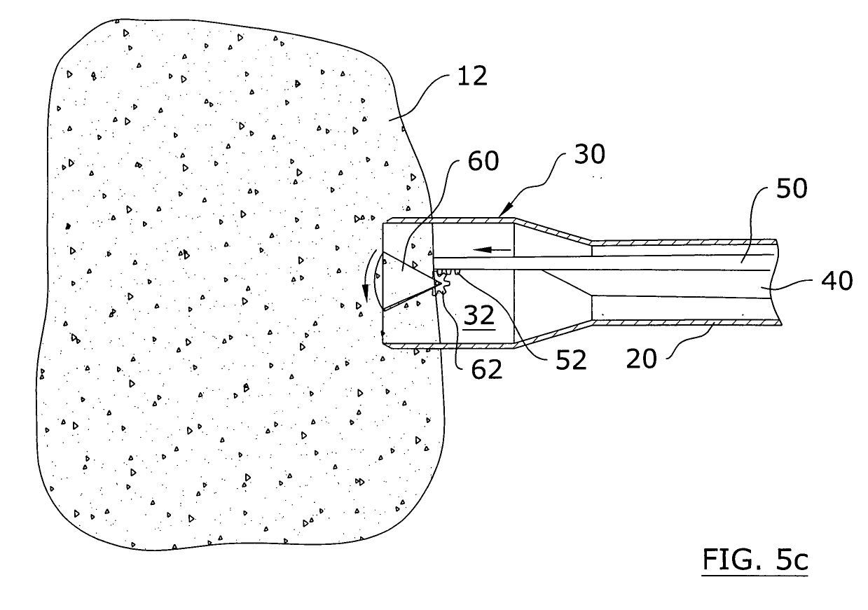

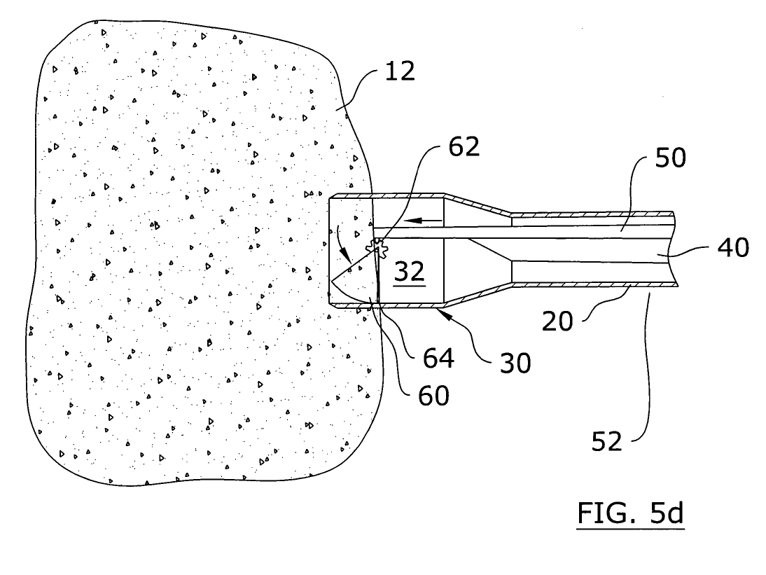

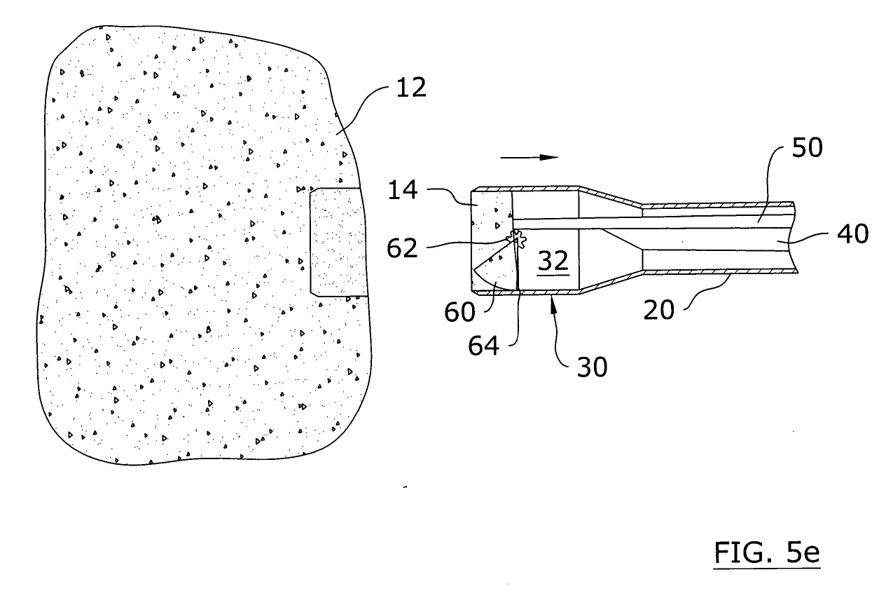

The arm member 50 is mechanically attached between the connector member 29 and the retrieval blade 60 for moving the retrieval blade 60 as shown in FIGS. 2 and 3 of the drawings. The arm member 50 preferably includes a plurality of teeth 52 for mechanically engaging the gear 62 on the retrieval blade 60 as best illustrates in FIGS. 4 through 5e of the drawings.

E. Operation of Invention

In use, the uterine cervix 12 is drenched with acetic acid which turns certain areas a white color representing dysplasia which need to be biopsied. The physician then inserts the punch 30 and housing 20 into the uterine cervix 12 guiding the punch 30 adjacent to an area of dysplasia. The punch 30 is concentrically aligned with the portion of the uterine cervix 12 that is desired to be sampled for medical testing as shown in FIG. 5a of the drawings. The physician then inserts the punch 30 into the inner wall of the uterine cervix 12 to a desired depth as shown in FIG. 5b of the drawings. The trigger member 28 is then drawn toward the handle 22 causing the arm member 50 to extend forwardly through the housing 20 thereby causing the retrieval blade 60 to rotate downwardly from the open position as shown in FIG. 5c of the drawings. As the retrieval blade 60 rotates, the blade edge 64 cuts through the tissue of the uterine cervix 12 to separate the biopsy tissue 14. Once the retrieval blade 60 is rotated to the closed position, the biopsy tissue 14 is either substantially or fully severed from the uterine cervix 12 as shown in FIG. 5d of the drawings. The physician then draws the present invention outwardly from the uterine cervix 12 wherein the retrieval blade 60 serves the purpose of grasping the biopsy tissue 14 to prevent removal of the biopsy tissue 14 as shown in FIG. 5e of the drawings. Once the punch 30 is removed from the uterine cervix 12, the trigger member 28 is allowed to return to the open position wherein the retrieval blade 60 extends upwardly to allow for the removal of the biopsy tissue 14 from the punch 30. The biopsy tissue 14 is then forwarded to the necessary medical testing facility. The present invention may be sanitized for repeated use thereafter. As can be appreciated, the present invention may be utilized for retrieving tissue samples from other portions of the human body.

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention, which is intended to be defined by the following claims (and their equivalents) in which all terms are meant in their broadest reasonable sense unless otherwise indicated. Any headings utilized within the description are for convenience only and have no legal or limiting effect.

We claim:

1. A cervical biopsy system, comprising:
a punch having a tubular structure, a cutting edge and an interior cavity; and
a retrieval blade movably positioned within said interior cavity of said punch for severing a biopsy tissue penetrated by said punch.

2. The cervical biopsy system of claim 1, including an elongated housing attached to said punch.

3. The cervical biopsy system of claim 2, including a handle attached to said elongated housing opposite of said punch.

4. The cervical biopsy system of claim 3, including a trigger member mechanically connected to said retrieval blade for manipulating said retrieval blade within said punch.

5. The cervical biopsy system of claim 4, wherein said trigger member is pivotally connected to said elongated housing near said handle.

6. The cervical biopsy system of claim 4, including a bias member positioned between said handle and said trigger member.

7. The cervical biopsy system of claim 4, wherein said trigger member includes a connector member extending past a pivot point and wherein said connector member is mechanically connected to said retrieval blade.

8. The cervical biopsy system of claim 7, including an arm member attached between said connector member and said retrieval blade for moving said retrieval blade.

9. The cervical biopsy system of claim 8, wherein said retrieval blade is rotatably attached within said punch.

10. The cervical biopsy system of claim 9, including a shaft member extending along a rotation axis of said retrieval blade.

11. The cervical biopsy system of claim 10, including a gear attached to said shaft member and wherein said arm member includes a plurality of teeth for mechanically engaging said gear.

12. The cervical biopsy system of claim 10, wherein said shaft member is substantially transverse with respect to a longitudinal axis of said punch.

13. The cervical biopsy system of claim 10, including a guide member attached within said housing for guiding said arm member along a longitudinal path.

14. The cervical biopsy system of claim 1, wherein said retrieval blade has a curved structure.

15. The cervical biopsy system of claim 1, wherein said retrieval blade includes a blade edge and wherein said blade edge is substantially C-shaped.

16. The cervical biopsy system of claim 1, including an elongated housing attached to said punch, a handle attached to said elongated housing opposite of said punch, and a thenar brace attached to said handle.

17. The cervical biopsy system of claim 1, wherein said punch has a circular cross sectional area.

18. The cervical biopsy system of claim 1, wherein said cutting edge of said punch has a circular shape.

19. A cervical biopsy system, comprising:
a punch having a tubular structure, a cutting edge and an interior cavity;
a retrieval blade rotatably positioned within said interior cavity of said punch for severing a biopsy tissue penetrated by said punch, wherein said retrieval blade has a curved C-shaped structure and wherein said retrieval blade includes a blade edge;
a shaft member extending along a rotation axis of said retrieval blade, wherein said shaft member is substantially transverse with respect to a longitudinal axis of said punch;
a guide member attached within said housing for guiding said arm member along a longitudinal path;
an elongated housing attached to said punch;
a handle attached to said elongated housing opposite of said punch;
a trigger member mechanically connected to said retrieval blade for manipulating said retrieval blade within said punch, wherein said trigger member is pivotally connected to said elongated housing near said handle and wherein said trigger member includes a connector member extending past a pivot point and wherein said connector member is mechanically connected to said retrieval blade;
a bias member positioned between said handle and said trigger member;
an arm member attached between said connector member and said retrieval blade for moving said retrieval blade; and
a gear attached to said shaft member and wherein said arm member includes a plurality of teeth for mechanically engaging said gear.

20. The cervical biopsy system of claim 19, wherein said punch has a circular cross sectional area and wherein said cutting edge of said punch has a circular shape.
